(19) United States
(12) Patent Application Publication
(10) Pub. No.: US 2014/0343421 A1
(43) Pub. Date: Nov. 20, 2014

(54) APPARATUS AND METHOD FOR SIMULTANEOUSLY PERFORMING RADIOTHERAPY AND HYPERTHERMIA THERAPY

(71) Applicants: Samsung Electronics Co., Ltd., Suwon-si (KR); Samsung Life Public Welfare Foundation, Seoul (KR)

(72) Inventors: Sang-hyun KIM, Hwaseong-si (KR); Young-sun KIM, Seoul (KR); Hyo-keun LIM, Seoul (KR); Won-chul BANG, Seongnam-si (KR)

(73) Assignees: SAMSUNG LIFE PUBLIC WELFARE FOUNDATION, Seoul (KR); SAMSUNG ELECTRONICS CO., LTD., Suwon-si (KR)

(21) Appl. No.: 14/277,206

(22) Filed: May 14, 2014

(30) Foreign Application Priority Data
May 14, 2013 (KR) 10-2013-0054644

(51) Int. Cl. A61N 7/02; A61B 8/08; A61B 5/01; A61G 13/02; A61B 19/00; A61B 5/00; A61B 6/03; A61B 5/055; A61N 5/10; A61B 8/00

(52) U.S. Cl. CPC A61N 7/02; A61N 5/10; A61B 8/08; A61B 5/01; A61B 8/4411; A61B 8/4494; A61B 19/2203; A61B 5/4836; A61B 6/032; A61B 5/055; A61G 13/02
USPC 600/438; 601/2; 600/2; 601/3; 600/439; 600/300; 600/427; 600/407; 600/411

(57) ABSTRACT

Provided are medical apparatuses for simultaneously performing radiotherapy and hyperthermia therapy and methods of treating an affected area. The medical apparatus includes a table where a subject is placed; a driving unit configured to move the table; a treatment device configured to treat an affected area of the subject; and an ultrasonic transducer configured to increase treatment efficiency of the affected area.

### RELATED APPLICATIONS

This application claims the benefit under 35 U.S.C. §119(a) of Korean Patent Application No. 10-2013-0054644, filed on May 14, 2013, in the Korean Intellectual Property Office, the entire disclosure of which is hereby incorporated by reference for all purposes.

### BACKGROUND

1. Field

The following description relates to a medical apparatus, and to a medical apparatus for simultaneously performing radiotherapy and hyperthermia therapy and a method of treating an affected area by using the same.

2. Description of Related Art

A treatment method using radiotherapy and hyperthermia therapy is known, and equipment for the treatment method has been developed. As apparatus for radiotherapy treatment and an apparatus using electromagnetic waves are combined, it may be difficult to control the equipment in real time, and it can be difficult to monitor a temperature. thus, it can be difficult to secure stability.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect there is provided a medical apparatus including a table where a subject is placed; a driving unit configured to move the table; a treatment device configured to treat an affected area of the subject; and an ultrasonic transducer configured to increase treatment efficiency of the affected area.

The ultrasonic transducer may be coupled to the treatment device, and may have a plurality of degrees of freedom.

The treatment device may include a radiation unit configured to emit radiation; a detection unit configured to detect the emitted radiation; and a supporting frame configured to support the radiation unit and the detection unit, and to guide the movement of the radiation unit and the detection unit.

The ultrasonic transducer may include a first ultrasonic transducer configured to increase a temperature of the affected area; and a second ultrasonic transducer configured to monitor at least one of a state of the affected area or the temperature of the affected area, the second ultrasonic transducer being removably attached to the first ultrasonic transducer.

The ultrasonic transducer may include a first ultrasonic transducer configured to increase a temperature of the affected area; and a second ultrasonic transducer configured to monitor at least one of a state of the affected area or the temperature of the affected area, the second ultrasonic transducer being removably attached to the first ultrasonic transducer.

The ultrasonic transducer may be disposed in the table.

The ultrasonic transducer may be attached to a separate robotic driving system.

A first end of the first ultrasonic transducer may be coupled to a first end of the second ultrasonic transducer, and a second end of the first ultrasonic transducer may be separate from a second end of the second ultrasonic transducer.

The medical apparatus may include an arm that may be movably attached to the supporting frame to be movable, wherein the ultrasonic transducer may be attached to an end of the arm distal from the supporting frame and may have a plurality of degrees of freedom.

The medical apparatus may include a monitoring device configured to monitor a treatment state of the affected area, wherein in response to the treatment device being coupled to the monitoring device, the treatment device may be provided in the monitoring device.

The monitoring device may be a computerized tomography (CT) device, an X-ray device, or a magnetic resonance imaging (MRI) device that is configured to monitor at least one of the treatment state or temperature of the affected area.

In response to the monitoring device being the MRI device, the ultrasonic transducer may be further configured to increase the temperature of the affected area.

The treatment device and the ultrasonic transducer may be movably disposed in a circular tunnel.

The ultrasonic transducer may include a telescopic arm movably attached to the supporting frame of the treatment device; a first ultrasonic transducer attached to an end of the arm distal from the supporting frame; a membrane configured to seal the first ultrasonic transducer and to provide a space between the membrane and the first ultrasonic transducer; and a second ultrasonic transducer disposed in the first ultrasonic transducer.

The ultrasonic transducer may include a telescopic arm movably attached to the supporting frame of the treatment device; a first ultrasonic transducer attached to an end of the arm distal from the supporting frame; and a second ultrasonic transducer attached to the arm through a bendable secondary arm.

The first ultrasonic transducer may be a high intensity focused ultrasonic (HIFU) device.

The medical apparatus may include a monitoring device configured to monitor a treatment state of the affected area, wherein the monitoring device may be connected to the treatment device.

The monitoring device may include a radiation unit configured to emit radiation; and a detection unit configured to detect the emitted radiation, and wherein the surface area of the detection unit is greater than the surface area of the radiation unit.

In another general aspect there is provided a method of treating a subject, the method including increasing a temperature of an affected area of the subject to a predetermined temperature; and performing radiotherapy on the affected area.

The method of treating a subject may include monitoring a state of the affected area after the radiotherapy.

The method of treating a subject may include monitoring a state of the affected area before and after increasing the temperature of the affected area.

The monitoring of the state of the affected area may be performed using a CT device, an X-ray device, or an MRI device before and after the radiotherapy is performed.

The increasing of the temperature may include increasing the temperature by irradiating ultrasonic wave on the affected area.

The increasing of the temperature may include increasing the temperature using an ultrasonic transducer or an MRI device before and after the radiotherapy is performed.

The temperature of the affected area may increase to the predetermined temperature before the radiotherapy is performed.

The temperature of the affected area may increase in response to the radiotherapy being performed.

The state of the affected area may include at least one of size depth, or position of the affected area.

The method of treating a subject may include monitoring a temperature of the affected area, and wherein the monitoring of the temperature of the affected area is performed using a an MRI device or an ultrasonic device.

A time of performing radiotherapy may be based on at least one of a size of the affected area, a tissue of the affected area, or a position of the affected area.

A time of performing radiotherapy may be predetermined.

The temperature of the affected area may increase to the predetermined temperature after the radiotherapy is performed.

In another general aspect there is provided a medical apparatus including a treatment device configured to treat an affected area of a subject, wherein the treatment device comprises a radiation unit configured to emit radiation, a detection unit configured to detect the emitted radiation, and a supporting frame configured to support the radiation unit and the detection unit, and to guide the movement of the radiation unit and the detection unit; and a ultrasonic transducer configured to increase a temperature of the affected area and to monitor at least one of a state of the affected area or the temperature of the affected area; wherein the ultrasonic transducer is movably attached to the supporting frame.

The ultrasonic transducer may include a telescopic arm movably attached to the supporting frame; a first ultrasonic transducer attached to an end of the arm distal from the supporting frame; a membrane configured to seal the first ultrasonic transducer and to provide a space between the membrane and the first ultrasonic transducer; and a second ultrasonic transducer disposed in the first ultrasonic transducer.

The medical apparatus may further comprise a monitoring device configured to monitor a treatment state of the affected area, wherein the monitoring device is a computerized tomography (CT) device, an X-ray device, or a magnetic resonance imaging (MRI) device.

Other features and aspects will be apparent from the following detailed description, the drawings, and the claims.

### BRIEF DESCRIPTION OF THE DRAWINGS

FIG. 1 is a diagram illustrating an example of a side view of a medical apparatus for simultaneously performing radiotherapy and hyperthermia therapy.

FIG. 2 is a diagram illustrating an example of an enlarged view of an ultrasonic transducer separated from the medical apparatus of FIG. 1.

FIG. 3 is a diagram illustrating an example of a right side view of the medical apparatus of FIG. 1.

FIG. 4 is a diagram illustrating an example of a modified ultrasonic transducer of FIG. 1.

FIG. 5 is a diagram illustrating an example of a right side view of a medical apparatus for simultaneously performing radiotherapy and hyperthermia therapy.

FIG. 6 is a diagram illustrating an example of a right side view of a medical apparatus for simultaneously performing radiotherapy and hyperthermia therapy.

FIG. 7 is a diagram illustrating an example of a side view of a medical apparatus for simultaneously performing radiotherapy and hyperthermia therapy.

FIG. 8 is a diagram illustrating an example of a side view of a medical apparatus.

FIG. 9 is a diagram illustrating an example of a front view of a monitoring apparatus of FIG. 8.

FIG. 10 is a diagram illustrating an example of a plan view of a medical apparatus.

FIG. 11 is a diagram illustrating an example of a left side view of the medical apparatus of FIG. 10.

FIG. 12 is a diagram illustrating an example of a side view of a medical apparatus.

FIG. 13 is a diagram illustrating an example of a cross-sectional view taken along a line 13-13′ of FIG. 12.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

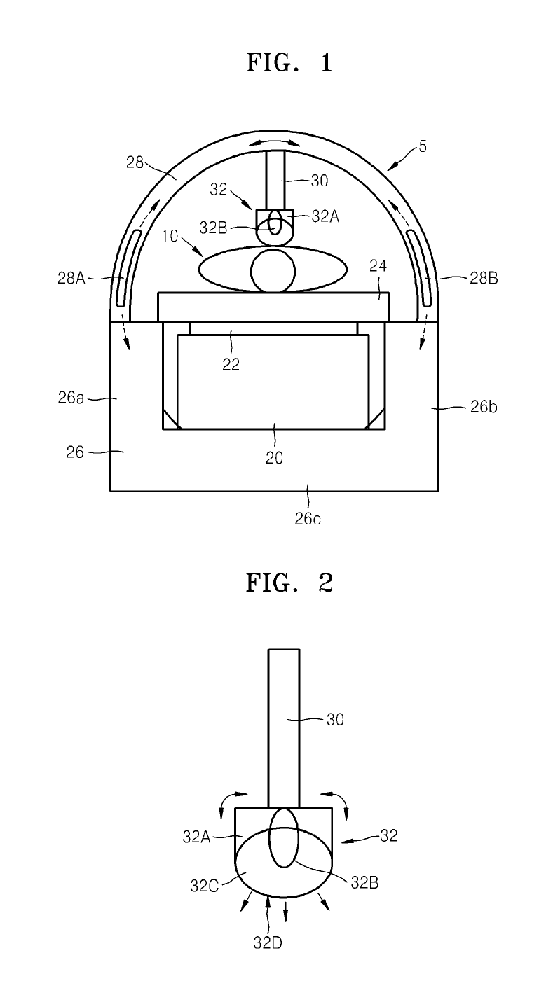

FIG. 1 is a diagram illustrating an example of a side view, when seen from a head of a subject 10, of a medical apparatus (hereinafter referred to as a first medical apparatus) for simultaneously performing radiotherapy and hyperthermia therapy.

Referring to FIG. 1, the first medical apparatus includes a radiation treatment unit (or a treatment apparatus) 5 and a table 24. A subject, for example, the subject 10, is placed on the table 24 when monitoring and treatment are being performed. A table driving unit 22 is disposed under the table 24. A first support 20, which supports the driving unit 22 and the table 24, is disposed under the table driving unit 22. A driving power generator, for example, a motor, of the driving unit 22 may be included in the first support 20. A second support 26 supports the radiation treatment unit 5. The second support 26 may be lower in position than the table 24. The second support 26 includes vertical sections 26a and 26b at both sides of a horizontal section 26c, which connects the vertical sections 26a and 26b. The second support 26 may be a single body. The radiation treatment unit 5 may include a circular tunnel or a supporting frame 28. Portions of the supporting frame 28 may be disposed in the second support 26. The table 24 is disposed within the supporting frame 28 and on the second support 26. The table 24 is separate from the supporting frame 28 by a predetermined distance. The supporting frame 28 may have a circular shape, such as for example, a circular ring shape, an open cylinder, a circular belt shape, or a circular donut shape that surrounds the subject 10 lying on the table 24. The supporting frame 28 and the subject 10 are separate from each other by a certain distance.

The radiation treatment unit 5 also includes a radiation emission unit 28A (hereinafter referred to as a radiation unit) and a detection unit 28B. The detection unit 28B may be, for example, an ion chamber. Radiation emitted from the radiation unit 28A may be detected by the detection unit 28B. The radiation unit 28A may emit radiation, such as, for example, X-rays, neutron rays, or gamma rays, and in addition, the radiation unit 28A may emit particle rays used for proton therapy. The detection unit 28B may include a sensing device (for example, a sensor) for sensing radiation or particle rays emitted from the radiation unit 28A. The radiation unit 28A and the detection unit 28B may be provided in the circular tunnel or in the supporting frame 28 and may be movable along the supporting frame 28 and in the supporting frame 28. Therefore, a position of each of the radiation unit 28A and detection unit 28B may be adjusted depending on a position of an affected area of the subject 10. The second support 26, the supporting frame 28, the radiation unit 28A, and the detection unit 28B may form the radiation treatment unit 5. An arm 30 is connected to the supporting frame 28. The arm 30 may project from the supporting frame 28 towards the table 24. The arm 30 may be movably coupled to the supporting frame 28. Therefore, the arm 30 may move along the supporting frame 28 while being attached to the supporting frame 28. Also, the arm 30 is telescopic, and thus may increase or decrease in length. The ultrasonic transducer 32 is connected to an end of the arm 30 distal from the supporting frame 28. Therefore, the ultrasonic transducer 32 may be applied to several body parts of a subject. Power may be supplied to the ultrasonic transducer 32 through the arm 30. The ultrasonic transducer 32 has a heating function that increases a temperature of an affected area. The ultrasonic transducer 32 also monitors both the affected area and an ambient temperature of the affected area in real time. The ultrasonic transducer 32 includes a first ultrasonic transducer 32A and a second ultrasonic transducer 32B. The second ultrasonic transducer 32B may be disposed in the first ultrasonic transducer 32A.

FIG. 2 is a diagram illustrating an example of an enlarged view of an ultrasonic transducer separated from the medical apparatus of FIG. 1. As illustrated in FIG. 2, the first and second ultrasonic transducers 32A and 32B are surrounded by a membrane 32D. The membrane 32D may be sealing for the surface of the first ultrasonic transducer 32A that is distal from the arm 30 and there may be some space between the membrane 32D and the first ultrasonic transducer 32A. The membrane 32D may be bonded to an edge of the first ultrasonic transducer 32A. The space between the membrane 32D and the first ultrasonic transducer 32A is filled with a liquid 32C. The liquid 32C may be, for example, water. The membrane 32D may directly contact a skin of the subject 10. Since membrane 32D is included in the ultrasonic transducer 32, the ultrasonic transducer 32 may be used without being affected by a curvature of a surface of the subject 10. The first ultrasonic transducer 32A may be an ultrasonic transducer (which increases a temperature of an affect area) for hyperthermia therapy or may be an ultrasonic transducer for treatment. The second ultrasonic transducer 32B may be a transducer for diagnosis or monitoring. The second ultrasonic transducer 32B is removably attached to the first ultrasonic transducer 32A so that it is attachable/detachable. Therefore, the second ultrasonic transducer 32B may be replaced, if needed.

The first ultrasonic transducer 32A may irradiate an ultrasonic wave on the affected area of the subject 10 to increase a temperature of the affected area to an appropriate temperature to enhance efficiency of treatment using the radiation treatment unit 5. For example, the first ultrasonic transducer 32A may increase the temperature of the affected area to about 40° C. to 45° C. The temperature of the affected area may be increased simultaneously with radiation treatment using the radiation treatment unit 5, or it may be increased before the radiation treatment. Alternatively, the temperature of the affected area may increase to be within a temperature range, and after the first ultrasonic transducer 32A is turned off, the radiation treatment may be performed. At this time, the second ultrasonic transducer 32B may monitor the temperature of the affected area in real time, and when the temperature of the affected area reaches a minimum value of the temperature range, the first ultrasonic transducer 32A may be used to increase the temperature of the affected area.

As described above, the first ultrasonic transducer 32A may be used as a type of heating device that increases the temperature of the affected area, but the first ultrasonic transducer 32A may be used as a high intensity focused ultrasonic (HIFU) apparatus that is used to treat the affected area. In this case, the first ultrasonic transducer 32A may be used to increase the temperature of the affected area to a temperature higher than the temperature range. For example, the first ultrasonic transducer 32A may increase the temperature of the affected area to 65° C. to 70° C., and thus, the affected area may be treated along with the radiation treatment.

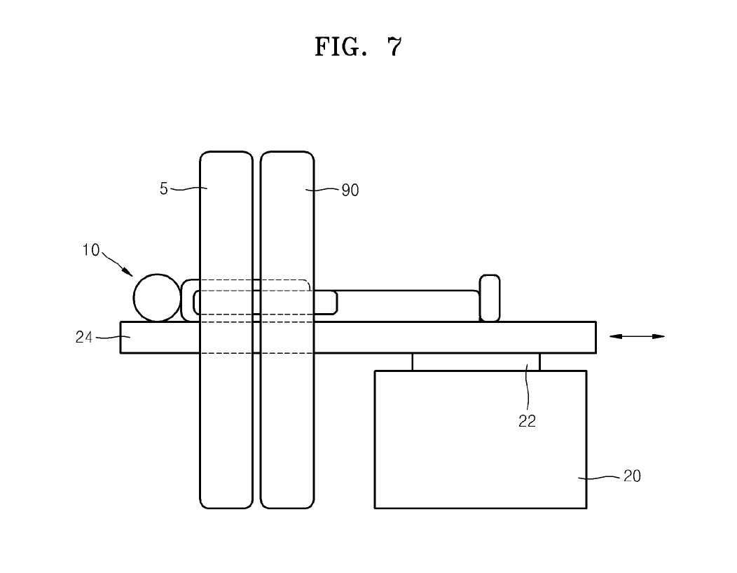

The first medical apparatus may further include a separate monitoring apparatus (for example, monitoring apparatuses illustrated in FIGS. 7 to 12). The separate monitoring apparatus may be used as an apparatus for monitoring a result of the radiation treatment. The separate monitoring apparatus may be a monitoring apparatus, such as, for example, a computerized tomography (CT) apparatus, an X-ray apparatus, or a magnetic resonance imaging (MRI) apparatus. A position, size, and/or state of an affected area of a subject are monitored using the separate monitoring apparatus before and after the radiation treatment.

Some of the separate monitoring apparatuses may be used to monitor a temperature of an affected area. For example, when a temperature of an affected area is increased by the first ultrasonic transducer 32A or by another method, a temperature and temperature distribution of the affected area may be monitored by an ultrasonic apparatus such as the second ultrasonic transducer 32B, or any of the separate monitoring apparatuses, such as the MRI apparatus.

The ultrasonic apparatus, such as the second ultrasonic transducer 32B, may also be used to monitor a position, size, and/or state of an affected area of a subject before and after the radiation treatment. Therefore, when the separate monitoring apparatus is included in the first medical apparatus, the second ultrasonic transducer 32B may or may not be provided. When the first ultrasonic transducer 32A is used as the HIFU apparatus, a treatment state of an affected area may be monitored using the separate monitoring apparatus. In this case, a temperature and temperature distribution of the affected area may be monitored by the MRI apparatus or the ultrasonic apparatus. Similarly to using the ultrasonic apparatus, the separate monitoring apparatus may perform monitoring in real time.

In monitoring an affected area before treating a subject, for example, when the radiation treatment and/or treatment using the first ultrasonic transducer 32A are/is carried out, the state (for example, a size, depth, position, etc.) of the affected area of the subject may be observed using the CT apparatus, the X-ray apparatus, the MRI apparatus, or the ultrasonic apparatus. In addition, a temperature and temperature distribution of the affected area may be monitored using the MRI apparatus or the ultrasonic apparatus. The hyperthermia treatment and radiation treatment of the affected area using the ultrasonic transducer may be performed based on the monitoring information about the state of the affected area. The separate monitoring apparatus will be described below.

Referring to FIG. 2, the ultrasonic transducer 32 mounted on the end of the arm 30 has a degree of freedom allowing it to move in all directions. For example, the ultrasonic transducer 32 may move left, right, upward, and downward at the end of the arm 30, and may rotate with the arm 30 as an axis. Therefore, any coupling that permits such a degree of freedom may be used when the ultrasonic transducer 32 is coupled to the arm 30.

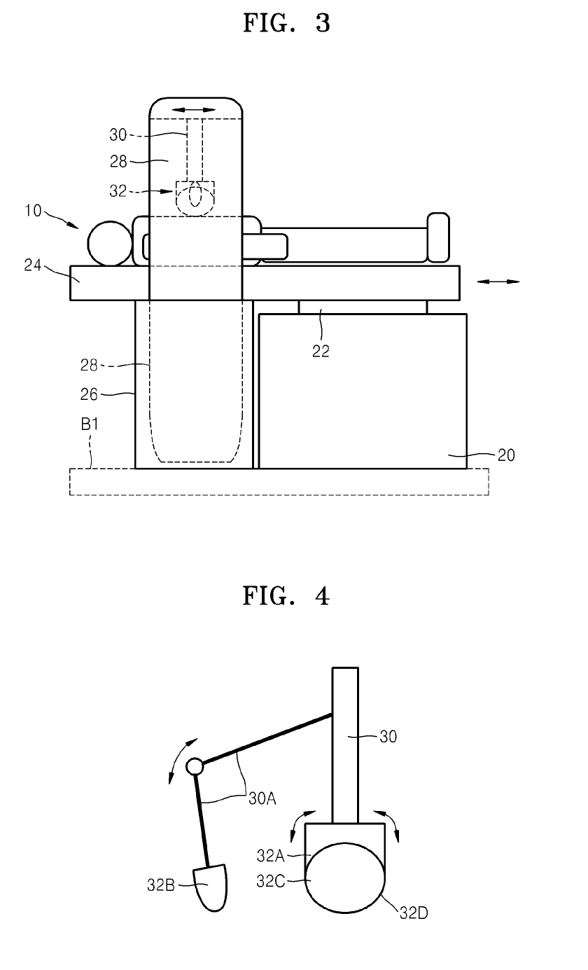

FIG. 3 is a diagram illustrating an example of a right side view of the first medical apparatus of FIG. 1. Referring to FIG. 3, the arm 30 may move left and right in the supporting frame 28. The arm 30 is connected to an internal surface of an upper end of the supporting frame 28. A portion of the supporting frame 28 is provided under the table 24 in the second support 26. The second support 26 and the supporting frame 28 are fixed, and the table 24 may be slidable and may be moved left and right by the driving unit 22.

The first and second supports 20 and 26 may be connected to each other and fixed. For example, as illustrated in FIG. 3, the first and second supports 20 and 26 may be fixed to a common base B1. A robotic driving ultrasonic transducer, which is described below, may also be mounted on the common base B1.

FIG. 4 is a diagram illustrating an example of a modified ultrasonic transducer 32 of FIG. 1. In FIG. 1, the first and second ultrasonic transducers 32A and 32B are implemented as one body. In FIG. 4, the second ultrasonic transducer 32B may be separate from the first ultrasonic transducer 32A, and the second ultrasonic transducer 32B is connected to the arm 30 through an assistant arm 30A.

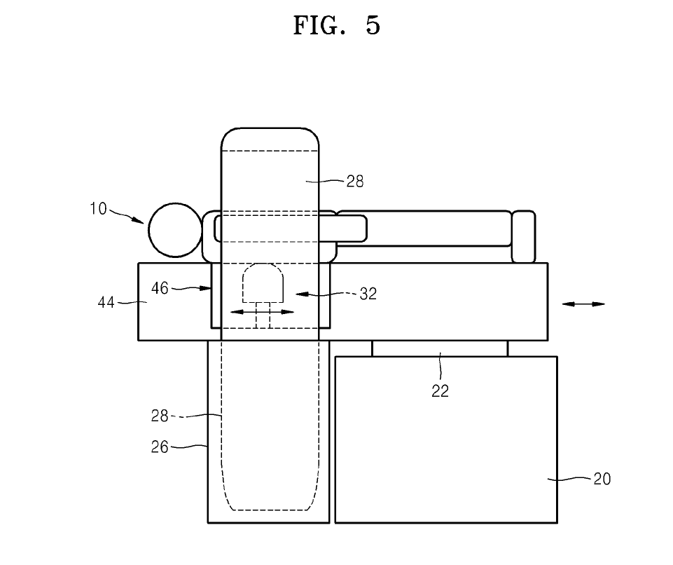

FIG. 5 is a diagram illustrating an example of a right side view of a medical apparatus (hereinafter referred to as a second medical apparatus) for simultaneously performing radiotherapy and hyperthermia therapy. The description of like elements of FIGS. 1-4 is applicable to the elements of FIG. 5, and thus will not be repeated here. Like reference numerals refer to like elements.

Referring to FIG. 5, the second medical apparatus includes an ultrasonic transducer 32 disposed in an internal space 46 of a table 44. Power may be supplied to the ultrasonic transducer 32 through the table 44 or through the table 44 and a driving unit. The ultrasonic transducer 32 may move in all four directions (front, back, left and right) in the internal space 46 of the table 44, and may move in a direction vertical to the ground. A width of the internal space 46 may be narrower or broader than that of the supporting frame 28. Other elements of the second medical apparatus may be similar to those of the first medical apparatus. The second medical apparatus may also be provided with a base, such as the base B1 of FIG. 3.

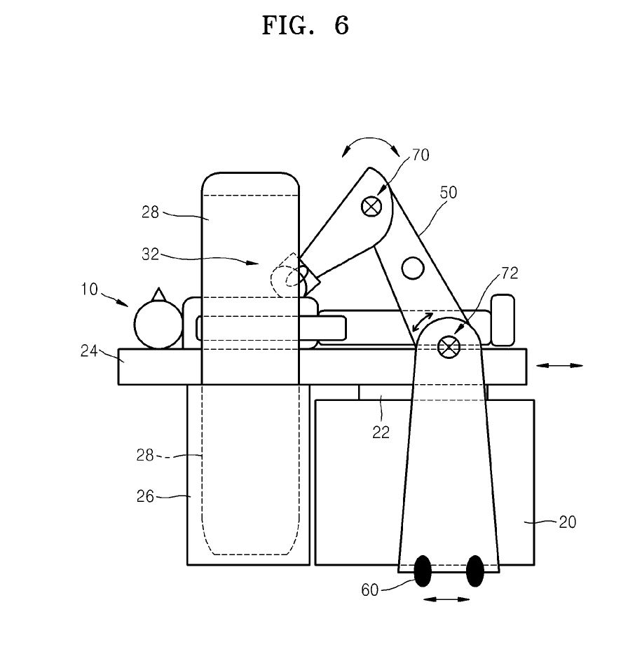

FIG. 6 is a diagram illustrating an example of a right side view of a medical apparatus (hereinafter referred to as a third medical apparatus) for simultaneously performing radiotherapy and hyperthermia therapy. The description of like elements of FIGS. 1-5 is applicable to the elements of FIG. 6, and thus will not be repeated here. Like reference numerals refer to like elements.

Referring to FIG. 6, the third medical apparatus provides an ultrasonic transducer 32 using a robotic driving system 50. The ultrasonic transducer 32 is mounted on an end of the robotic driving system. The robotic driving system 50 may move on the ground using a set of wheel 60. The robotic driving system 50 may move independently or the movement of the robotic driving system 50 may be coordinated with the movement of a table 24. The robotic driving system 50 may use a multi-joint robot, and include a plurality of rotation shafts 70 and 72. The ultrasonic transducer 32 mounted on the robotic driving system 50 has a degree of freedom similar to the first medical apparatus. The robotic driving system 50 may be mounted on the base B1 described above with reference to FIG. 3, in which case the wheel 60 may not be required. In another non-exhaustive example, the multi-joint robot may be mounted on the table 24, and the ultrasonic transducer 32 may be mounted on a joint end of the multi-joint robot.

FIG. 7 is a diagram illustrating an example of a side view of a medical apparatus (hereinafter referred to as a fourth medical apparatus) for simultaneously performing radiotherapy and hyperthermia therapy. The description of like elements of FIGS. 1-6 is applicable to the elements of FIG. 7, and thus will not be repeated here. Like reference numerals refer to like elements.

Referring to FIG. 7, the fourth medical apparatus includes a radiation treatment unit 5 and a first monitoring apparatus 90. The first monitoring apparatus 90 may be an apparatus, such as, for example a CT apparatus. As illustrated in FIG. 7, the radiation treatment unit 5 and the first monitoring apparatus 90 may be placed next to each other horizontally. The radiation treatment unit 5 and the first monitoring apparatus 90 may be separate from each other, or may be coupled to each other. The radiation treatment unit 5 and the first monitoring apparatus 90 may be disposed on the same axis. By using the first monitoring apparatus 90, a state of an affected area of a subject 10 may be observed before radiation treatment is performed, and a treatment state of the affected area may be observed during treatment or after the treatment. When a temperature of the affected area is increased by using the ultrasonic transducer 32, a temperature and temperature distribution of the affected area may be monitored using an ultrasonic apparatus such as the second ultrasonic transducer 32B (see FIG. 1). The monitoring may be performed in real time. When the temperature of the affected area reaches a temperature suitable for radiation treatment, a radiation treatment process that irradiates radiation onto the affected area by using the radiation treatment unit 5 may be performed. The temperature of the affected area may increase after or during the radiation treatment. The radiation treatment process may be performed for a predetermined time, and an execution time may be changed according to a position and size of the affected area, namely, a tissue and size of the subject. When the ultrasonic transducer 32 is used as the HIFU apparatus, the first monitoring apparatus 90 may be used to observe the state of the affected area after or during the HIFU treatment. In the treatment and monitoring process, the table 24 may move, or the radiation treatment unit 5 and the first monitoring apparatus 90 may move.

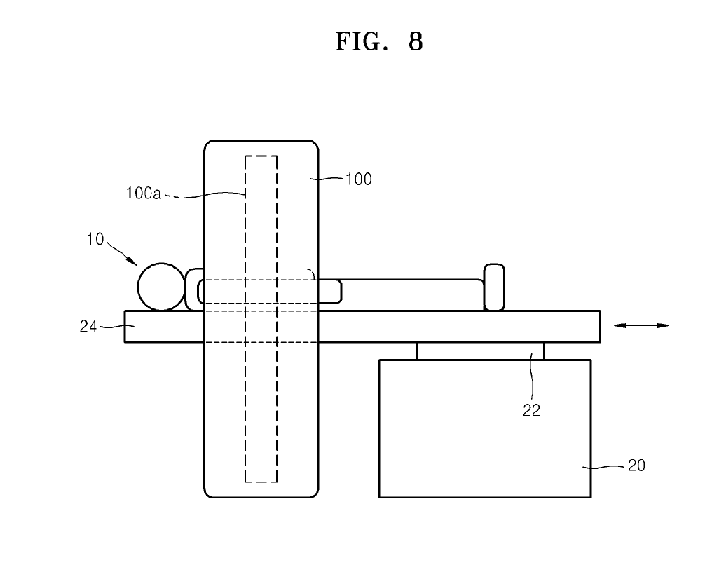

FIG. 8 is a diagram illustrating an example of a side view of a medical apparatus (hereinafter referred to as a fifth medical apparatus). The description of like elements of FIGS. 1-7 is applicable to the elements of FIG. 8, and thus will not be repeated here. Like reference numerals refer to like elements. In the fifth medical apparatus, a radiotherapy and hyperthermia therapy apparatus is provided in a monitoring apparatus.

Referring to FIG. 8, the fifth medical apparatus includes a second monitoring apparatus 100, which may perform the same function as the first monitoring apparatus 90 of FIG. 7. The second monitoring apparatus 100 may be, for example, a CT apparatus. A treatment apparatus 100a may be provided in the second monitoring apparatus 100. The treatment apparatus 100a may include the radiation treatment unit 5 of the first medical apparatus of FIG. 1. Therefore, the treatment apparatus 100a may perform a function of the radiation treatment unit 5.

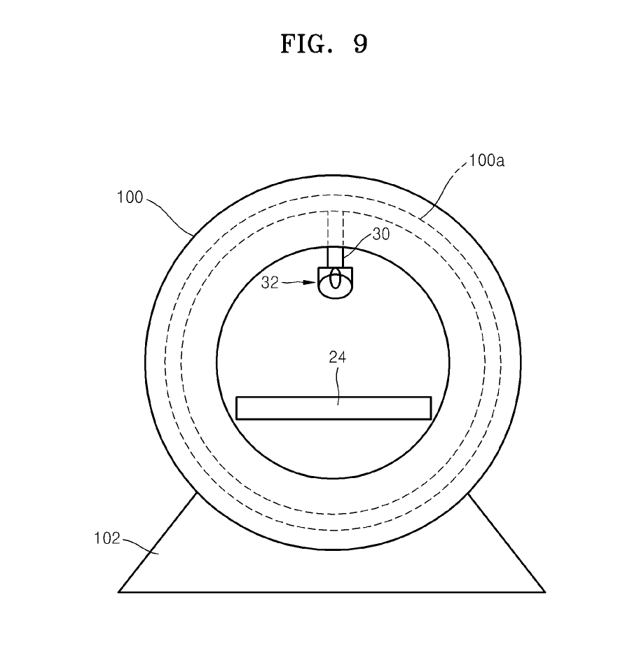

FIG. 9 is a diagram illustrating an example of a front view of the second monitoring apparatus 100 of FIG. 8. Referring to FIG. 9, the second monitoring apparatus 100 may have a circular shape, such as for example, a circular ring shape, a circular tunnel, an open cylinder, a circular belt shape, or a circular donut shape. The table 24 is placed in the interior opening of the second monitoring apparatus 100. The treatment apparatus 100a is included in the second monitoring apparatus 100. The medical apparatus 100a is provided along a circular structure of the second monitoring apparatus 100. Therefore, the medical apparatus 100a has a circular shape, such as, for example, a circular ring shape, a circular tunnel, an open cylinder, a circular belt shape, or a circular donut shape. The ultrasonic transducer 32 is connected to the medical apparatus 100a. The ultrasonic transducer 32 protrudes outside the second monitoring apparatus 100 towards the table 24. The connection between the ultrasonic transducer 32 and the medical apparatus 100a may be similar to the radiation treatment unit 5 and ultrasonic transducer 32 of the first medical apparatus. A support member 102 supports the second monitoring apparatus 100.

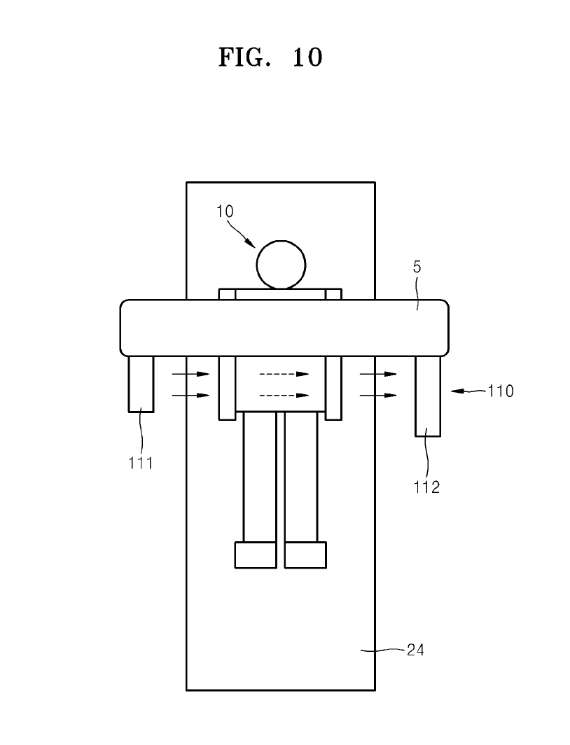

FIG. 10 is a diagram illustrating an example of a plan view of a medical apparatus (hereinafter referred to as a sixth medical apparatus). The description of like elements of FIGS. 1-9 is applicable to the elements of FIG. 10, and thus will not be repeated here. Like reference numerals refer to like elements.

Referring to FIG. 10, the sixth medical apparatus includes a radiation treatment unit 5 and a third monitoring apparatus 110. In the example shown in FIG. 10, the third monitoring apparatus 110 is connected to the radiation treatment unit 5, but may be provided independently from the radiation treatment unit 5. The third monitoring apparatus 110 may be an apparatus, such as, for example, an X-ray apparatus. The third monitoring apparatus 110 may irradiate X-rays onto an affected area to monitor a position, size, pre-treatment state, treatment state, or post-treatment state of the affected area. Therefore, the third monitoring apparatus 110 may have the same function as the first monitoring apparatus 90 of FIG. 7. The third monitoring apparatus 110 includes an emission unit 111 and a reception unit 112. X-rays are emitted from the emission unit 111. The emitted X-rays are received by the reception unit 112 after being transmitted through the affected area. The emission unit 110 and the reception unit 112 may be positioned to face each other. The emission unit 110 may be connected to one side of the radiation treatment unit 5, and the reception unit 112 may be connected to the other side of the radiation treatment unit 5. Alternatively, the emission unit 110 may be disposed at one side (for example, the left) of a subject 10, and the reception unit 112 may be disposed at the other side (for example, the right) of the subject 10. In the sixth medical apparatus, the ultrasonic transducer 32 may be included in the radiation treatment unit 5 as illustrated in FIG. 1. The function of the ultrasonic transducer 32 may be the same as that described in relation to FIG. 1.

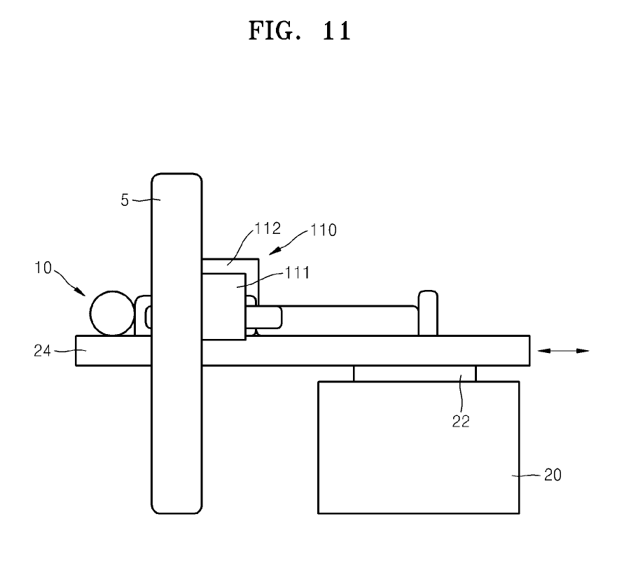

FIG. 11 is a diagram illustrating an example of a left side view of the sixth medical apparatus of FIG. 10. In the non-exhaustive example shown in FIG. 11, an area of an X-ray reception unit 112 is broader than that of an X-ray emission unit 110. The affected area may be treated using the radiation treatment unit 5, and then a treatment state of the affected area may be observed using the third monitoring apparatus 110. After the radiation treatment using the radiation treatment unit 5 is completed, the table 24 may move in order for the affected area to be disposed between the emission unit 110 and the reception unit 112. The radiation treatment using the radiation treatment unit 5 may be performed at several stages. In this case, observation (i.e., movement of the table 24) of the state of the affected area using the third monitoring apparatus 110 may be performed each time when a stage of the radiation treatment is completed. Although not shown in FIGS. 10 and 11, the ultrasonic transducer 32 may be included in the radiation treatment unit 5 as illustrated in FIG. 1.

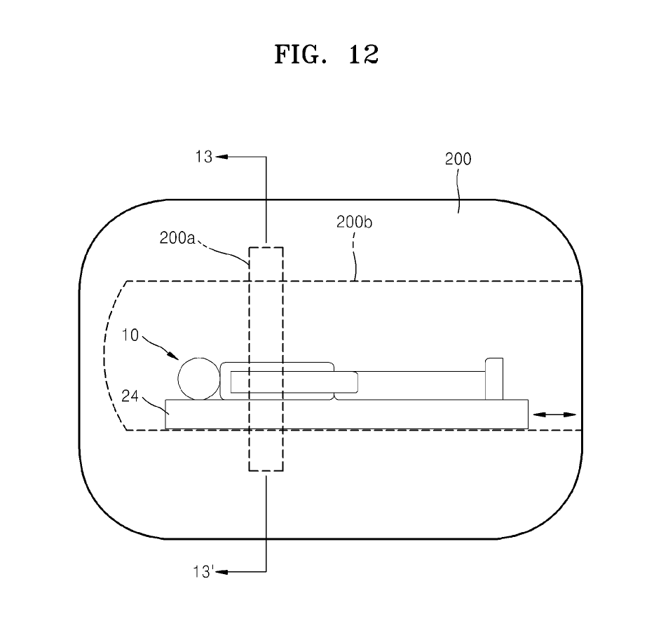

FIG. 12 is a diagram illustrating an example of a side view of a medical apparatus (hereinafter referred to as a seventh medical apparatus). The description of like elements of FIGS. 1-11 is applicable to the elements of FIG. 12, and thus will not be repeated here. Like reference numerals refer to like elements.

Referring to FIG. 12, the seventh medical apparatus includes a fourth monitoring apparatus 200 and a treatment apparatus 200a. The treatment apparatus 200a may be provided in the fourth monitoring apparatus 200, i.e., the fourth monitoring apparatus 200 and the treatment apparatus 200a may have a one body. The fourth monitoring apparatus 200 may be as apparatus, such as, for example, an MRI apparatus that may monitor a treatment state and a temperature of the affected area.

The fourth monitoring apparatus 200 may be used to observe the state of the affected area before, during, or after treatment using the treatment apparatus 200a. A function of the fourth monitoring apparatus 200 may be the same as the first monitoring apparatus 90 of FIG. 7. The fourth monitoring apparatus 200 may be used to monitor a temperature of the affected area before and after the treatment. The treatment apparatus 200a may be a radiation treatment apparatus, and for example, may include the radiation treatment unit 5 of FIG. 1. The fourth monitoring apparatus 200 has a tunnel 200b of a predetermined length. The length of the tunnel 200b may be equal to or longer than the length of table 24. Treatment of a subject 10 and monitoring of the state of the affected area are performed when the subject 10 is in the tunnel 200b. The treatment and the monitoring of the state of the affected area may be performed in real time. Also, the treatment and the monitoring of the state of the affected area may be performed sequentially. For example, information on the position and state of the affected area of the subject 10 may be obtained using the fourth monitoring apparatus 200, and then the affected area may be treated using the treatment apparatus 200a on the basis of the obtained information. If the treatment is performed at several stages, the state of the affected area may be observed using the fourth monitoring apparatus 200 when each stage is completed. An amount and irradiation time of treatment rays irradiated from the treatment apparatus 200a onto the affected area may be changed depending on a position and type of the affected area. In the fourth monitoring apparatus 200, the treatment apparatus 200a may be separately moved or driven.

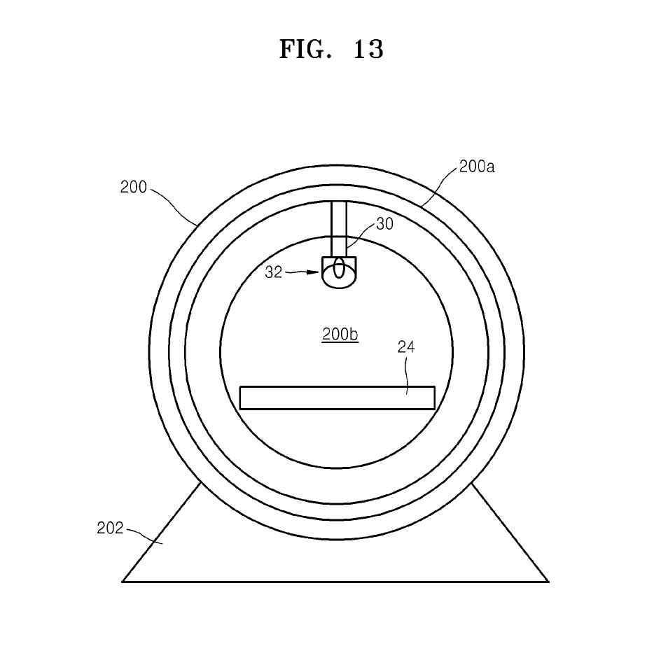

FIG. 13 is a diagram illustrating an example of a cross-sectional view taken along a line 13-13′ of FIG. 12. Referring to FIG. 13, the fourth monitoring apparatus 200 has a circular shape, such as for example, a circular ring shape, a circular tunnel, an open cylinder, a circular belt shape, or a circular donut shape. The treatment apparatus 200a is provided in the fourth monitoring apparatus 200, and thus may have a circular shape, such as for example, a circular ring shape, a circular tunnel, an open cylinder, a circular belt shape, or a circular donut shape. An ultrasonic transducer 32 is connected to the treatment apparatus 200a. The ultrasonic transducer 32 may be configured similar to the ultrasonic transducer of FIG. 1, and have the similar motion characteristic. The ultrasonic transducer 32 may protrude outside the fourth monitoring apparatus 200 towards the table 24. When the ultrasonic transducer 32 is used as the HIFU apparatus, the fourth monitoring apparatus 200 may be used to monitor the state of the affected area after treatment using the HIFU apparatus. When the temperature of the affected area in increased through the ultrasonic transducer 32, the temperature and temperature distribution of the affected area may be monitored using the fourth monitoring apparatus 200. When the fourth monitoring apparatus 200 is the MRI apparatus, the ultrasonic transducer 32 may include only a first ultrasonic transducer 32A that increases the temperature of the affected area.

Like support member 102 of FIG. 9, in FIG. 13, a support member 202 supports the fourth monitoring apparatus 200. Although not shown, the treatment apparatus 200a may be provided at an entrance of the tunnel of the fourth monitoring apparatus 200. In this case, the affected area of the subject 10 may be treated at the entrance of the tunnel of the fourth monitoring apparatus 200 by using the treatment apparatus 200a, and the subject 10 may be transferred into the tunnel 200b of the fourth monitoring apparatus 200 to monitor the treatment state of the affected area.

The processes, functions, and methods described above can be written as a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more non-transitory computer readable recording mediums. The non-transitory computer readable recording medium may include any data storage device that can store data that can be thereafter read by a computer system or processing device. Examples of the non-transitory computer readable recording medium include read-only memory (ROM), random-access memory (RAM), Compact Disc Read-only Memory (CD-ROMs), magnetic tapes, USBs, floppy disks, hard disks, optical recording media (e.g., CD-ROMs, or DVDs), and PC interfaces (e.g., PCI, PCI-express, WiFi, etc.). In addition, functional programs, codes, and code segments for accomplishing the example disclosed herein can be construed by programmers skilled in the art based on the flow diagrams and block diagrams of the figures and their corresponding descriptions as provided herein.

The apparatuses and units described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A medical apparatus comprising:
a table where a subject is placed;
a driving unit configured to move the table;
a treatment device configured to treat an affected area of the subject; and
an ultrasonic transducer configured to increase treatment efficiency of the affected area.

2. The medical apparatus of claim 1, wherein the ultrasonic transducer is coupled to the treatment device, and has a plurality of degrees of freedom.

3. The medical apparatus of claim 1, wherein the treatment device comprises:
a radiation unit configured to emit radiation;
a detection unit configured to detect the emitted radiation; and
a supporting frame configured to support the radiation unit and the detection unit, and to guide the movement of the radiation unit and the detection unit.

4. The medical apparatus of claim 1, wherein the ultrasonic transducer comprises:
a first ultrasonic transducer configured to increase a temperature of the affected area; and
a second ultrasonic transducer configured to monitor at least one of a state of the affected area or the temperature of the affected area, the second ultrasonic transducer being removably attached to the first ultrasonic transducer.

5. The medical apparatus of claim 2, wherein the ultrasonic transducer comprises:
a first ultrasonic transducer configured to increase a temperature of the affected area; and
a second ultrasonic transducer configured to monitor at least one of a state of the affected area or the temperature of the affected area, the second ultrasonic transducer being removably attached to the first ultrasonic transducer.

6. The medical apparatus of claim 1, wherein the ultrasonic transducer is disposed in the table.

7. The medical apparatus of claim 1, wherein the ultrasonic transducer is attached to a separate robotic driving system.

8. The medical apparatus of claim 4, wherein a first end of the first ultrasonic transducer is coupled to a first end of the second ultrasonic transducer, and a second end of the first ultrasonic transducer is separate from a second end of the second ultrasonic transducer.

9. The medical apparatus of claim 3, further comprising an arm that is movably attached to the supporting frame to be movable,
wherein the ultrasonic transducer is attached to an end of the arm distal from the supporting frame and has a plurality of degrees of freedom.

10. The medical apparatus of claim 1, further comprising a monitoring device configured to monitor a treatment state of the affected area,
wherein in response to the treatment device being coupled to the monitoring device, the treatment device is provided in the monitoring device.

11. The medical apparatus of claim 10, wherein the monitoring device is a computerized tomography (CT) device, an X-ray device, or a magnetic resonance imaging (MRI) device that is configured to monitor at least one of the treatment state or temperature of the affected area.

12. The medical apparatus of claim 11, wherein in response to the monitoring device being the MRI device, the ultrasonic transducer is further configured to increase the temperature of the affected area.

13. The medical apparatus of claim 1, wherein the treatment device and the ultrasonic transducer are movably disposed in a circular tunnel.

14. The medical apparatus of claim 3, wherein the ultrasonic transducer comprises:
a telescopic arm movably attached to the supporting frame of the treatment device;
a first ultrasonic transducer attached to an end of the arm distal from the supporting frame;
a membrane configured to seal the first ultrasonic transducer and to provide a space between the membrane and the first ultrasonic transducer; and
a second ultrasonic transducer disposed in the first ultrasonic transducer.

15. The medical apparatus of claim 3, wherein the ultrasonic transducer comprises:
a telescopic arm movably attached to the supporting frame of the treatment device;
a first ultrasonic transducer attached to an end of the arm distal from the supporting frame; and
a second ultrasonic transducer attached to the arm through a bendable secondary arm.

16. The medical apparatus of claim 3, wherein the first ultrasonic transducer is a high intensity focused ultrasonic (HIFU) device.

17. The medical apparatus of claim 10, further comprising a monitoring device configured to monitor a treatment state of the affected area, wherein the monitoring device is connected to the treatment device.

18. The medical apparatus of claim 17, wherein the monitoring device further comprises:
a radiation unit configured to emit radiation; and
a detection unit configured to detect the emitted radiation, and wherein the surface area of the detection unit is greater than the surface area of the radiation unit.

19. A method of treating a subject, the method comprising:
increasing a temperature of an affected area of the subject to a predetermined temperature; and
performing radiotherapy on the affected area.

20. The method of claim 19, further comprising, monitoring a state of the affected area after the radiotherapy.

21. The method of claim 19, further comprising, monitoring a state of the affected area before and after increasing the temperature of the affected area.

22. The method of claim 21, wherein the monitoring of the state of the affected area is performed using a CT device, an X-ray device, or an MRI device before and after the radiotherapy is performed.

23. The method of claim 19, wherein the increasing of the temperature comprises increasing the temperature by irradiating ultrasonic wave on the affected area.

24. The method of claim 21, wherein the increasing of the temperature comprises increasing the temperature using an ultrasonic transducer or an MRI device before and after the radiotherapy is performed.

25. The method of claim 19, wherein the temperature of the affected area increases to the predetermined temperature before the radiotherapy is performed.

26. The method of claim 19, wherein the temperature of the affected area increases in response to the radiotherapy being performed.

27. The method of claim 20, wherein the state of the affected area comprises at least one of size depth, or position of the affected area.

28. The method of claim 19, further comprising monitoring a temperature of the affected area, and wherein the monitoring of the temperature of the affected area is performed using an MRI device or an ultrasonic device.

29. The method of claim 19, wherein a time of performing radiotherapy is based on at least one of a size of the affected area, a tissue of the affected area, or a position of the affected area.

30. The method of claim 19, wherein a time of performing radiotherapy is predetermined.

31. The method of claim 19, wherein the temperature of the affected area increases to the predetermined temperature after the radiotherapy is performed.

32. A medical apparatus comprising:
a treatment device configured to treat an affected area of a subject, wherein the treatment device comprises a radiation unit configured to emit radiation, a detection unit configured to detect the emitted radiation, and a supporting frame configured to support the radiation unit and the detection unit, and to guide the movement of the radiation unit and the detection unit; and
a ultrasonic transducer configured to increase a temperature of the affected area and to monitor at least one of a state of the affected area or the temperature of the affected area; wherein the ultrasonic transducer is movably attached to the supporting frame.

33. The medical apparatus of claim 32, wherein the ultrasonic transducer comprises:
a telescopic arm movably attached to the supporting frame;
a first ultrasonic transducer attached to an end of the arm distal from the supporting frame;
a membrane configured to seal the first ultrasonic transducer and to provide a space between the membrane and the first ultrasonic transducer; and
a second ultrasonic transducer disposed in the first ultrasonic transducer.

34. The medical apparatus of claim 32, further comprising a monitoring device configured to monitor a treatment state of the affected area, wherein the monitoring device is a computerized tomography (CT) device, an X-ray device, or a magnetic resonance imaging (MRI) device.
